(19) United States
(12) Patent Application Publication
(10) Pub. No.: US 2005/0276764 A1
(43) Pub. Date: Dec. 15, 2005

(54) Topical use of bis-arylimidazo[1,2-a]thiolane derivatives

(75) Inventors: Ludger Kolbe, Dohren (DE); Uwe Pfannenbecker, Hamburg (DE); Inge Kruse, Hamburg (DE); Tobias Sokolowski, Hamburg (DE); Jeannine Immeyer, Egestorf-Sahrendorf (DE); Karen Tom Dieck, Hamburg (DE); Gerd Dannhardt, Mainz (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 11/147,117

(22) Filed: Jun. 6, 2005

Related U.S. Application Data
(63) Continuation of application No. PCT/EP03/50865, filed on Nov. 21, 2003.

(30) Foreign Application Priority Data
Dec. 5, 2002 (DE) 102 56 881.2

(51) Int. Cl. A61K 31/429; A61K 7/42; A61K 7/15

(52) U.S. Cl. 424/59; 514/366

(57) ABSTRACT

The invention is a topical, anti-inflammatory medical, cosmetic, or dermatological preparation comprising at least one compound of the formula (DKH) 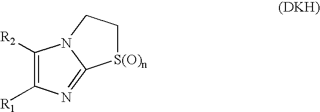 wherein n is 1 or 2; and
R₁ and R₂, independently of each other, are selected from the group consisting of phenyl, 2-toly, 3-tolyl, 4-tolyl, 2-methoxyphenyl, 3-methoxyphenyl, and 4-methoxyphenyl. The invention also includes methods for treating or preventing itchiness, hyperreactive skin conditions, sensitive skin, photostress, impure skin, the effects of UV radiation, and irritation comprising applying said preparation to the skin.

### CROSS-REFERENCE TO RELATED APPLICATIONS

This is a continuation of PCT/EP2003/050865, filed Nov. 21, 2003, which is incorporated herein by reference in its entirety, and also claims the benefit of German Priority Application No. 102 56 881.2, filed Dec. 5, 2002.

### FIELD OF THE INVENTION

The present invention concerns new uses of definite bis-arylimidazo[1,2-a]thiolane derivatives.

### BACKGROUND OF THE INVENTION

Bis-arylimidazoles have been extensively examined in the past with the aim of finding substances with anti-inflammatory effects. This resulted in success in many cases. A large part of these efforts were nevertheless oriented towards medical preparations for oral administration, but not for application on the skin. Part of the reason for this lies in the fact that many cyclooxygenase inhibitors, which also include the bis-arylimidazoles described here, are known for their photocytotoxic properties (Acta Derm Venereol 1996,76:33740).

Preparations with anti-inflammatory effect play a significant role in cosmetics and dermatology. Such preparations have different fields of application, for example soothing shaving burn.

The growth of facial hair is stimulated in growing men by increased production of male hormones during puberty. Hormonal disturbances in women can also lead to a form of facial hair which is nevertheless significantly less extensive than male facial hair growth in its development.

Shaving the face or other parts of the body covered with hair (such as the legs, armpit or pubic area) can be motivated by several constraints—e.g. of a religious or cultural nature; in the most straightforward case, hair growth is undesired by the person concerned for purely cosmetic reasons.

Shaving is carried out either dry or wet. The development of new mechanical and electrical wet and dry shaving techniques nowadays enables a safe and thorough removal of the (facial) hair. With wet shaving, chemical aids—for example in the form of shaving gels, soaps or foams—are generally essential.

These are required in order to soften the (facial) hair and hence minimize the effort required for cutting through—and consequently the unpleasant pulling on the hair shaft. Softening the (facial) hair is achieved through water absorption which is enabled by increasing the pH value of the hair. Wet shave agents therefore generally contain soap orfattyacid salts whose pH value lies in the range from 8-10. Products for wet shaving therefore produce a typical skin feeling which occurs after application. The skin feels dry and rough to the touch. This skin feeling is also referred to as a “squeaky-feeling” in the cosmetic industry and is extremely unpopular among consumers.

Cosmetic agents are also frequently recommended for dry shaving as well, so as to achieve as close a shave as possible, i.e. to cut the (facial) hair as closely to the skin surface as possible.

The skin parts affected by shaving can nevertheless not only be irritated by shaving aids, the mechanical irritation caused by shaving itself can also represent a stress to the skin which can lead to an unpleasant skin feeling (the so-called “shaving burn”).

A task of the present invention was therefore to find cosmetic or dermatological preparations which are more effective in reducing the post-reactions of the skin to the (mechanical) irritation caused by shaving.

Moreover, the present invention further concerns preparations with an extremely low so-called “stinging potential”.

The problem of “sensitive skin” affects an increasing number of adults and children. Sensitive skin refers to a combination of various symptoms, such as hyperreactive and intolerant skin. However, atopic skin can also be subsumed among these. These skin conditions are often, albeit not strictly correctly, referred to as “allergic” skin by those affected. Although an allergic illness can lead to symptoms of sensitive skin, the phenomenon of “sensitive skin” is not restricted to individuals suffering from an allergy.

The skin, in particular the epidermis, is especially prone to external influences as a barrier organ of the human organism. According to current scientific understanding, the skin represents an immunological organ which, as an immunocompetent peripheral compartment, plays a unique role in inductive, effective and regulative immunoprocesses of the entire organ.

The epidermis is richly endowed with nerves and nerve endings such as Vater-Pacini lamellar corpuscles, Merkel cell-neurite complexes and free nerve endings for the sense of pain, cold, heat and itching.

For individuals with sensitive, tender or injured skin, a neurosensory phenomenon characterized by stinging can be observed. This “sensitive skin” differs fundamentally from “dry skin” with thickened and hardened strata cornea.

Typical reactions of “stinging” with sensitive skin are reddening, tightening and burning of the skin as well as itching.

Itchiness with atopic skin as well as itchiness with skin disorders are to be regarded as a neurosensory phenomenon.

“Stinging” phenomena can be regarded as disturbances to be treated cosmetically. More significant itching (in particular with pronounced skin itchiness occurring during atopic disorders), on the other hand, can also be designated as a serious dermatological disturbance.

Typical disruptive neurosensory phenomena associated with the terms “stinging” or “sensitive skin” are skin reddening, tingling, prickling, tightness and burning of the skin as well as itchiness. They can be caused by stimulant environmental conditions—e.g. massage, the effect of (wash-active) surfactants, climatic influence such as sun, cold, dryness, but also heat, radiant heat and UV radiation, e.g. the sun.

In the “Journal of the Society of Cosmetic Chemists” 28, pp.197-209 (May 1977), P. J. Frosch and A. M. Kligman describe a method for estimating the “stinging potential” of topically administered substances. Lactic acid and pyruvic acid are typically used as positive substances here. In measurements according to this method, however, amino acids, in particular glycine, were also determined as active in neurosensory terms (such substances are referred as to “stingers”).

According to previous findings, the presence of such a form of sensitivity to very definite substances varies from individual to individual. This means that a person who experiences “stinging effects” on contact with a substance will very likely experience these again on every subsequent contact. Contact with other “stingers” can, however, occur perfectly normally without any reaction.

Many individuals who are more or less sensitive also have to endure erythematous skin symptoms when using certain deodorants or antiperspirants.

Erythematous skin symptoms also occur as accompanying symptoms with certain skin diseases or irregularities. As an example, the typical skin rash in the symptoms of acne is regularly characterized by more or less significant reddening.

The paper Chemical Abstract, Volume 111 No. 97243c of 11.9.1989 discloses 2-(4,5-diphenylimidazol)-(2-pyridylmethyl)-sulfide, but not the methoxy- or chloroaryl derivatives.

The paper JP 1040467 A discloses heterocyclene-alkylene-thio-substituted Phenylimidazole, but not any 4,5-bis(p-methoxyphenyl)-imidazole derivatives.

The paper Chemical Abstract, Volume 71 No.112863f of 8.12.1969 discloses 2-(4,5-diphenylimidazol)-(2-pyridylethyl)-sulfide, but not any 4,5-bis(p-methoxyphenyl)-imidazole derivatives.

The paper Acta Chim. Budapest 1969, 61(1), Pages 69-77 discloses substituted benzimidazoles in its abstract, but not any arylimidazo[1,2-a]thiolane derivatives.

The paper Chemical Abstract, Volume 78, No. 72002k of 19.3.1973 discloses phenyl-substituted 4,5-diphenylimidazole derivatives, as well as cyclic and 4,5-bis(p-methoxyphenyl)imidazole derivatives; however, no aryl imidazo[1,2-a]thiolane derivatives appear to be present.

The published patent application DE 2823197 discloses imidazole derivatives, but not any arylalkyl-sulfur-substituted imidazole derivatives.

The published patent application WO 91/10662 discloses imidazole derivatives which are linear substituted at the 2-position, but not any arylimidazo[1,2-a]thiolane derivatives.

The published patent application EP372445 discloses imidazole carbamates and ureas.

The published patent application DE 19842833 discloses 5-heteroaryl-imidazole derivatives.

The published patent application WO 95/00501 discloses various heterocyclic cyclooxygenase inhibitors, but not any arylimidazo[1,2-a]thiolane derivatives.

None of these published patent applications discloses more detailed information on the problem concerning the phototoxicity of such compounds with topical application. It has nevertheless been revealed that this property significantly limits the use of these compounds in topical preparations.

### SUMMARY OF THE INVENTION

On the basis hereof, the task was to find anti-inflammatory, topical medical preparations as well as cosmetic and/or dermatological preparations which are characterized by low phototoxicity at a sufficient level of effectiveness and which, in particular, provide long-lasting care for skin which has been subjected to photostress.

It has been revealed in a manner unforeseeable to the skilled expert that topical, anti-inflammatory medical preparations containing compounds with the formula (DKH) 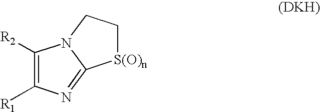
where n has the values 1 or 2 and R₁ and R₂ represent, independently of one another, the grouping phenyl, 2-tolyl, 3-tolyl, 4-tolyl, 2-methoxyphenyl, 3-methoxyphenyl, 4-methoxyphenyl remedy the shortcomings of the prior art. Furthermore, topical, anti-inflammatory cosmetic and/or dermatological preparations containing compounds with the formula (DKH) where n has the values 1 or 2 and R₁ and R₂ represent, independently of one another, the grouping phenyl, 2-tolyl, 3-tolyl, 4-tolyl, 2-methoxyphenyl, 3-methoxyphenyl, 4-methoxyphenyl, also remedy the shortcomings of the prior art.

These preparations exhibit the following advantages in comparison to the preparations of the prior art:

The substances can be stably incorporated in formulations and exhibit good penetration to the site of action (epidermis, dermis, endothelium) after application to the skin.

It was also surprising that cosmetic or dermatological formulations according to the invention are characterized by low phototoxicity, an indispensable prerequisite for the use of active substances in cosmetics which are applied to skin exposed to light, which is very often known to be the case.

The user also expects these preparations according to the invention to be well tolerated, this applies in particular to cosmetics with anti-inflammatory active substances such as those described here, as they are applied as required to sensitive skin, skin irritated by shaving or sunburn or skin subjected to other such stresses. In these cases, it is also especially important that these preparations are tolerated without problems. Moreover, these preparations should nevertheless also be appealing cosmetically. The preparations according to the invention fulfill all these requirements.

It was also ascertained that it is preferable if R₁ and R₂ represent the grouping 4-methoxyphenyl. It is particularly preferred if n=1.

It is also preferred if the compound with the formula (DKH) is present in concentrations of 10 to 0.0001% by weight, especially preferred from 0.001 to 1% by weight and most particularly preferred from 0.01 to 0.1% by weight.

### BRIEF DESCRIPTION OF THE DRAWINGS

FIG. 1 is a graph illustrating the photocytotoxicity of 4,5-bis(4-methoxyphenyl)-imidazo[1,2-a]thiolane-1-oxide and 4,5-bis(4-methoxyphenyl)-imidazo[1,2-a]thiolane-1-dioxide in comparison to 4,5-bis(4-methoxyphenyl)-imidazo[1,2-a]thiolane according to standard protocol “3T3 NRU Phototoxicity Assay” of the COLIPA Validation Ring Study of 1997.

FIG. 2 is a graph illustrating the PGE2 content in fibroblasts as a function of concentration for LPS, diclofenac, DKH 29, DKH 29 sulfone, DKH 29 sulfoxide, and a control group.

FIG. 3 is a graph illustrating the LTB4 content in granulocytes as a function of concentration for fMLP, BayX1005, DKH 29, DKH 29 sulfone, DKH 29 sulfoxide, and a control.

### DETAILED DESCRIPTION OF THE PREFERRED EMBODIMENTS

Such preparations according to the invention are preferably used whereby the anti-inflammatory effect is directed against psoriasis, atopic eczema, acne, rosacea, allergic and irritative contact dermatitis, sunburn and shaving burn, diaper dermatitis, seborrhoeic dermatitis, sun allergy (polymorphic light eruption, Mallorca acne) and actinic keratoses. Particularly preferred are preparations according to the invention used for the treatment and prophylaxis of itchiness, hyperreactive skin conditions, sensitive skin, skin subjected to photostress and impure skin.

A most particularly preferred use of such preparations involves application of the preparation to the sun-exposed skin.

The invention also comprises the use of compounds with the formula (DKH) for the production of topical, anti-inflammatory medical preparations, where n has the values 1 or 2 and R₁ and R₂ represent, independently of one another, the grouping phenyl, 2-tolyl, 3-tolyl, 4-tolyl, 2-methoxyphenyl, 3-methoxyphenyl, 4-methoxyphenyl as well as the use of compounds with the formula (DKH) for the production of topical; anti-inflammatory cosmetic or dermatological preparations, where n has the values 1 or 2 and R₁ and R₂ represent, independently of one another, the grouping phenyl, 2-tolyl, 3-tolyl, 4-tolyl, 2-methoxyphenyl, 3-methoxyphenyl, 4-methoxyphenyl.

All these preparations and applications remedy the deficiencies of the prior art by manifesting further advantages in comparison to the preparations of the prior art.

The formulations for the purpose of the present invention are extremely satisfactory preparations in every respect, which are distinguished by a long-lasting skin-care effect. It was not foreseeable to the skilled expert that the formulations used according to the invention would provide improved care for skin subjected to photostress and shaving stress, would more effectively better reduce the post-reactions of the skin to the effects of UV radiation and the (mechanical) irritation caused by shaving, would more effectively alleviate skin irritated by sunbathing and shaving, would cause mild sunburn and shaving burn to subside more rapidly, would more effectively promote skin smoothing, would be distinguished by improved skincare effects, and would exhibit sensory properties, such as ease of application onto the skin or penetration into the skin, than the preparations of the prior art.

It was also surprising that the preparations for the purpose of the present invention also reduce the post-reactions of the skin to the effects of UV radiation and to the (mechanical) irritation caused by shaving, if they are used (i.e. applied to the skin) before or while sunbathing or shaving.

The invention is, of course, not restricted to preparations which are applied after sunburn or shaving, but also naturally comprises all cosmetic and dermatological applications for which a stress-alleviating effect is desired or advantageous. The abovementioned positive effect of the formulations according to the invention similarly applies for skin subjected to photostress and skin irritated by shaving.

It is very particularly preferred to use 4,5-bis(4-methoxyphenyl)-imidazo[1,2-a]thiolane-1-oxide as well as 4,5-bis(4-methoxyphenyl)-imidazo[1,2-a]thiolane-1-dioxide in preparations according to the invention. Photocytotoxicity tests have surprisingly revealed that these substances exhibit a considerably reduced phototoxicity in comparison to the non-oxidized comparative compound 4.5-bis(4-methoxyphenyl)-imidazo[1,2-a]thiolane. At the same time, it was demonstrated that the production of leukotriene B4 in granulocytes and the production of prostaglandin E2 in fibroblasts is considerably reduced, and that the substances are consequently very effective.

Skin moisturizing agents which can be used advantageously are glycerol, chitosan, Fucogel, propylene glycol, dipropylene glycol, butylene glycol, mannitol, lactic acid, sodium pyrrolidonecarboxylic acid, hyaluronic acid, salts of the given acids, as well as glycine, urea and salts of metals of the first and second main group.

Glycerol, lactic acid, butylene glycol, urea, hyaluronic acid are particularly suitable.

The content of skin moisturizing agents is advantageously 3% by weight to 60% by weight, preferably 4 to 50% by weight, in particular 5 to 40% by weight, based on the total weight of the preparations.

It is also advantageous in the sense of the present invention to create cosmetic and dermatological preparations whose main purpose is not protection against sunlight, but which nevertheless contain a content of UV protection substances. Thus, for example, UV-A and/or UV-B filter substances are usually incorporated into day creams or makeup products. UV protection substances, like antioxidants and, if desired, preservatives, also represent effective protection of the preparations themselves against spoilage. Also favourable are cosmetic and dermatological preparations which are present in the form of a sunscreen agent.

Accordingly, the preparations in the sense of the present invention preferably contain at least one UV-A and/or UV-B filter substance.

The formulations may, but do not necessarily, optionally also contain one or more organic and/or inorganic pigments as UV filter substances, which may be present in the water phase and/or the oil phase.

Preferred inorganic pigments are metal oxides and/or other metal compounds which are sparingly soluble or insoluble in water, in particular oxides of titanium (TiO₂), zinc (ZnO), iron (e.g. Fe₂O₃), zirconium (ZrO₂), silicon (SiO₂), manganese (e.g. MnO), aluminium (Al₂O₃), cerium (e.g. Ce₂O₃), mixed oxides of the corresponding metals, and mixtures of such oxides, as well as the sulphate of barium (BaSO₄).

The titanium dioxide pigments may be present either in the crystal modification rutile, or else in the form of anatase and may, in the sense of the present invention, be advantageously surface-treated (“coated”), the intention being to form or retain, for example, a hydrophilic, amphiphilic or hydrophobic character. This surface treatment can involve providing the pigments with a thin hydrophilic and/or hydrophobic inorganic and/or organic layer by processes known per se. The various surface coatings can also comprise water in the sense of the present invention.

Described coated and uncoated titanium dioxides can also be used in the sense of the present invention in the form of commercially available oily or aqueous predispersions. Dispersion auxiliaries and/or solubilization promoters may advantageously be added to these predispersions.

The titanium dioxides according to the invention are characterized by a primary particle size between 10 nm to 150 nm.

| | | Additional | |
|---|---|---|---|
| | | constituents | |
| | | of the | |
| Trade name | Coating | predispersion | Manufacturer |
| MT-100TV | Aluminum hydroxide | — | Tayca |
| | Stearic acid | | Corporation |
| MT-100Z | Aluminum hydroxide | — | Tayca |
| | Stearic acid | | Corporation |
| MT-100F | Stearic acid | — | Tayca |
| | Iron oxide | | Corporation |
| MT-500SAS | Alumina, silica | — | Tayca |
| | silicone | | Corporation |
| MT-100AQ | Silica | — | Tayca |
| | Aluminum hydroxide | | Corporation |
| | Alginic acid | | |
| Eusolex T-2000 | Alumina | — | Merck KgaA |
| | simethicones | | |
| Eusolex TS | Alumina, stearic acid | — | Merck KgaA |
| Titanium dioxide | None | — | Degussa |
| P25 | | | |
| Titanium dioxide | Octyltrimethylsilane | — | Degussa |
| T805 (Uvinul | | | |
| TiO₂) | | | |
| UV-Titan X170 | Alumina | — | Kemira |
| | Dimethicones | | |
| UV-Titan X161 | Alumina, silica | — | Kemira |
| | stearic acid | | |
| Tioveil AQ 10PG | Alumina | Water | Solaveil |
| | silica | Propylene | Uniquema |
| | glycol | | |
| Mirasun TiW 60 | Alumina | Water | Rhone-Poulenc |
| | silica | | |

In the sense of the present invention, particularly preferred titanium dioxides are MT-100 Z and MT-100 TV from Tayca Corporation, Eusolex T-2000 and Eusolex TS from Merck and Titanium Dioxide T 805 from Degussa.

In the sense of the present invention, zinc oxides can also be used in the form of commercially available oily or aqueous predispersions. Zinc oxide particles suitable according to the invention and predispersions of zinc oxide particles are characterized by a primary particle size of <300 nm and are available under the following trade names from the companies listed:

| Trade name | Coating | Manufacturer |
|---|---|---|
| Z-Cote HPI | 2% Dimethicones | BASF |
| Z-Cote | / | BASF |
| ZnO NDM | 5% Dimethicones | H&R |
| MZ 707M | 7% Dimethicones | M. Tayca Corp. |
| Nanox 500 | / | Elementis |
| ZnO Neutral | / | H&R |

Particularly preferred zinc oxides in the sense of the invention are Z-Cote HP1 from BASF and Zinc Oxide NDM from Haarmann & Reimer. The total amount of one or more inorganic pigments in the finished cosmetic preparation is advantageously chosen from the range 0.1% by weight to 25% by weight, preferably 0.5% by weight to 18% by weight.

An advantageous organic pigment in the sense of the present invention is 2,2′-methylenebis(6-(2H-benzotriazol-2-yl)-4-(1,1,3,3-tetramethylbutyl)phenol) [INCI: Bisoctyltriazole], which is characterized by the chemical structural formula 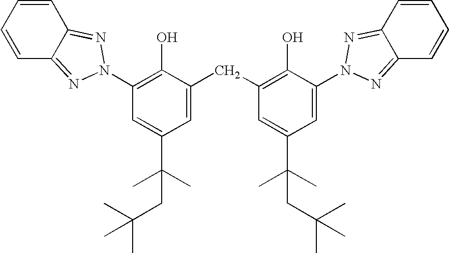
and is available under the trade name Tinosorb® M from CIBA-Chemikalien GmbH.

Advantageous UV-A filter substances in the sense of the present invention are dibenzoylmethane derivatives, in particular 4-(tert-butyl)-4′-methoxydibenzoylmethane (CAS No. 70356-091), which is sold by Givaudan under the name PARSOL® 1789 and by Merck under the trade name EUSOLEX® 9020. Further advantageous UV-A filter substances are phenylene-1,4-bis(2-benzimidazyl)-3,3′-5,5′-tetrasulphonic acid 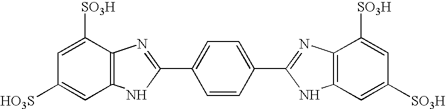
and its salts, particularly the corresponding sodium, potassium or triethanolammonium salts, in particular phenylene-1,4-bis(2-benzimidazyl)- -3,3′-5,5′-tetrasulphonic bis-sodium salt 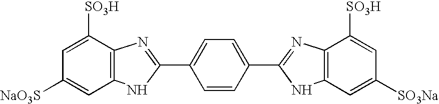
with the INCI name Bisimidazylate, which is available, for example, under the trade name Neo Heliopan AP from Haarmann & Reimer.

Also advantageous are 1,4-di(2-oxo-10-sulpho-3-bornylidenemethyl )benzene and salts thereof (in particular the corresponding 10-sulphato compounds, in particular the corresponding sodium, potassium or triethanolammonium salt), which is also referred to as benzene-1,4-di(2-oxo-3-bornylidenemethyl-10-sulphonic acid) and is characterized by the following structure:

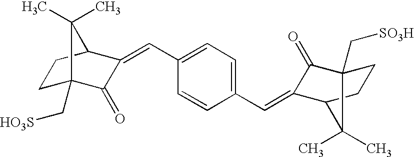

Further advantageous UV-A filter substances are hydroxybenzophenones which are characterized by the following structural formula: 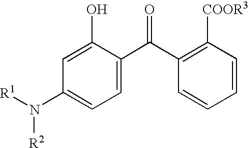
where

R¹ and R², independently of one another, are hydrogen, C,-C₂₀-alkyl, C₃-C,₀-cycloalkyl or C₃-C₁₀-cycloalkenyl where the substituents R′ and R² together with the nitrogen atom to which they are bonded, can form a 5-membered or 6-membered ring and R³ is a C,-C₂₀-alkyl radical.

A particularly advantageous hydroxybenzophenone in the sense of the present invention is hexyl 2-(4′-diethylamino-2′-hydroxybenzoyl)benzoic acid hexylester (also: aminobenzophenone), which is characterized by the following structure: 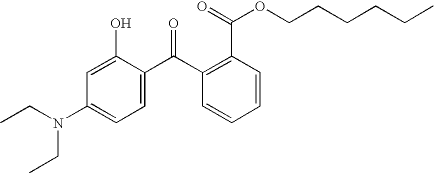
and is available under the trade name Uvinul A Plus from BASF.

Advantageous UV filter substances in the sense of the present invention are also so-called broadband filters, i.e. filter substances which absorb both UV-A and also UV-B radiation.

Advantageous broadband filters or UV-B filter substances are, for example, bisresorcinyltriazine derivatives having the following structure: 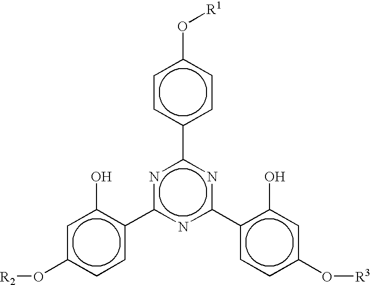
where R¹, R² and R³, independently of one another, are chosen from the group of branched and unbranched alkyl groups having 1 to 10 carbon atoms, or are a single hydrogen atom. Particular preference is given to 2,4-bis{[4-(2-ethylhexyloxy)-2-hydroxy]-phenyl}-6-(4-methoxyphenyl)-1,3,5-triazine (INCI: Aniso Triazine), which is available under the trade name TINOSORB® S from CIBA-Chemikalien GmbH.

In the sense of the present invention, particularly advantageous preparations which are characterized by high or very high UV-A protection preferably contain two or more UV-A and/or broadband filters, in particular dibenzoylmethane derivatives [for example 4-(tert-butyl)-4′-methoxydibenzoylmethane], benzotriazole derivatives [for example 2,2′-methylenebis(6-(2H-benzotriazol-2-yl)-4-(1,1,3,3-tetramethylbutyl)phenol)], phenylene-1,4-bis(2-benzimidazyl)-3,3′-5,5′-tetrasulphonic acid and/or its salts, 1,4-di(2-oxo-10-sulpho-3-bornylidenemethyl)benzene and/or salts thereof and/or 2,4-bis{[4-(2-ethylhexyloxy)-2-hydroxy]phenyl}-6-(4-methoxyphenyl)-1,3,5-triazine, in each case individually or in any combinations with one another.

Other UV filter substances, which have the structural formula 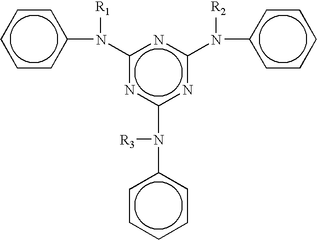
are also advantageous UV filter substances in the sense of the present invention, for example the s-triazine derivatives described in European laid-open specification EP 570 838 A1, whose chemical structure is expressed by the generic formula 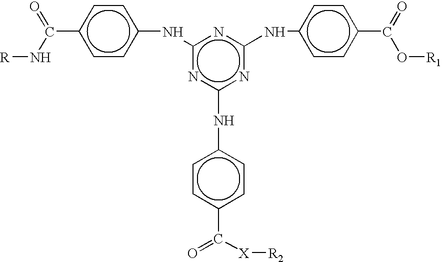
where

R is a branched or unbranched C,-C₁₈-alkyl radical, a C5-C12-cycloalkyl radical, optionally substituted by one or more C,-C₄-alkyl groups,

X is an oxygen atom or an NH group,

R₁ is a branched or unbranched C,-C₁₈-alkyl radical, a C5-C12-cycloalkyl radical, optionally substituted by one or more C,-C₄-alkyl groups, or a hydrogen atom, an alkali metal atom, an ammonium group or a group of the formula 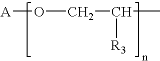
where

A is a branched or unbranched C,-C₁₈-alkyl radical, a C₅-C₁₂-cycloalkyl or aryl radical, optionally substituted by one or more C,-C₄-alkyl groups,

R₃ is a hydrogen atom or a methyl group,

n is a number from 1 to 10,

R₂ is a branched or unbranched C,-C₁₈-alkyl radical, a C₅-C₁₂-cycloalkyl radical, optionally substituted by one or more C,-C₄-alkyl groups, when X is the NH group, and a branched or unbranched C,-C₁₈-alkyl radical, a C₅-C₁₂-cycloalkyl radical, optionally substituted by one or more C,-C₄-akyl groups, or a hydrogen atom, an alkali metal atom, an ammonium group or a group of the formula 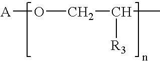
where

A is a branched or unbranched C,-C₁₈-alkyl radical, a C₅-C₁₂-cycloalkyl or aryl radical, optionally substituted by one or more C,-C₄-alkyl groups,

R₃ is a hydrogen atom or a methyl group,

n is a number from 1 to 10, when X is an oxygen atom.

A particularly preferred UV filter substance in the sense of the present invention is also an unsymmetrically substituted s-triazine, the chemical structure of which is expressed by the formula 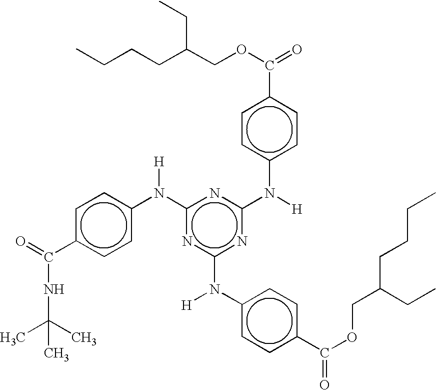
and which is also referred to below as dioctylbutylamidotriazone (INCI: Dioctylbutamidotriazone), and is available under the trade name UVASORB HEB from Sigma 3V.

Also advantageous in the sense of the present invention is a symmetrically substituted s-triazine, tris(2-ethylhexyl) 4,4′,4″-(1,3,5-triazine-2,4,6-triyltriimino)trisbenzoate, synonym: 2,4,6-tris[anilino-(p-carbo-2′-ethyl-1′-hexyloxy)]-1,3,5-triazine (INCI: Octyl Triazone), which is marketed by BASF Aktiengesellschaft under the trade name UVINUL® T 150.

European laid-open specification 775 698 also describes bisresorcinyltriazine derivatives to be preferably used, the chemical structure of which is expressed by the generic formula 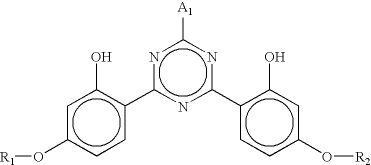
where R₁, R₂ and A represent very different organic radicals.

Also advantageous in the sense of the present invention are 2,4-bis{[4-(3-sulphonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethyl hexyloxy)-2-hydroxy]phenyl}-6-[4-(2-methoxyethyl-carboxyl )phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethyl-hexyloxy)-2-hydroxy]phenyl}-6-( 1-methylpyrrol-2-yl )-1,3,5-triazine, 2,4-bis{[4-tris(trimethyl-siloxysilyl propyloxy)-2-hydroxy]-phenyl}-6-(4-methoxyphenyl)-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

An advantageous broadband filter in the sense of the present invention is 2,2′-methylenebis(6-(2H-benzotriazol-2-yl)-4-(1,1,3,3-tetramethylbutyl)phenol), which is characterized by the chemical structural formula 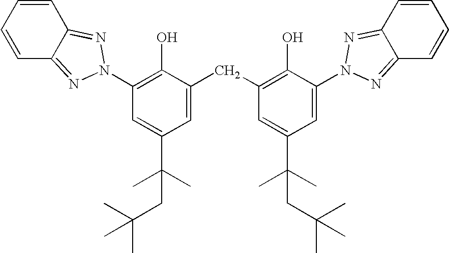
and is available under the trade name TINOSORB® M from CIBA-Chemikalien GmbH.

Another advantageous broadband filter in the sense of the present invention is 2-(2H-benzotriazol-2-yl)-4-methyl-6-[2-methyl-3-[1,3,3,3-tetramethyl-1-[(trimethylsilyl)-oxy]disiloxanyl]propyl]phenol (CAS No.: 155633-54-8) having the INCI name Drometrizole Trisiloxane, which is characterized by the chemical structural formula

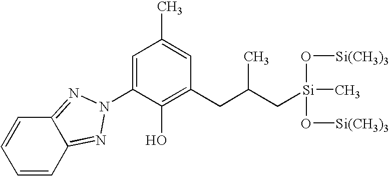

The UV-B and/or broadband filters can be oil-soluble or water-soluble. Examples of advantageous oil-soluble UV-B and/or broadband filter substances are:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate;

2,4,6-trianilino(p-carbo-2′-ethyl-1′-hexyloxy)-1,3,5-triazine;

esters of benzalmalonic acid, preferably di(2-ethylhexyl) 4-methoxybenzalmalonate;

esters of cinnamic acid, preferably 2-ethylhexyl 4-methoxycinnamate, isopentyl 4-methoxycinnamate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone; and

UV filters bonded to polymers.

Examples of advantageous water-soluble UV-B and/or broadband filter substances are:

salts of 2-phenylbenzimidazole-5-sulphonic acid, such as its sodium, potassium or its triethanolammonium salt, and also the sulphonic acid itself;

sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl)-sulphonic acid and salts thereof.

Particularly advantageous UV filter substances which are liquid at room temperature in the sense of the present invention are homomenthyl salicylate (INCI: Homosalate), 2-ethylhexyl 2-hydroxybenzoate (2-ethylhexyl salicylate, INCI: Octyl Salicylate), 4-isopropylbenzyl salicylate and esters of cinnamic acid, preferably (2-ethylhexyl) 4-methoxycinnamate (INCI: Octyl Methoxycinnamate) and isopentyl 4-methoxycinnamate (INCI: Isoamyl p-Methoxycinnamate), 3-(4-(2,2-bisethoxycarbonylvinyl)-phenoxy)propenyl)methoxysiloxane/dimethylsiloxane copolymer (INCI: Dimethicodiethyl-benzalmalonate) which is available, for example, under the trade name PARSOL® SLX from Hoffmann La Roche.

A further photoprotective filter substance which can be used advantageously according to the invention is ethylhexyl 2-cyano-3,3-diphenylacrylate (octocrylene), which is available from BASF under the name UVINUL® N 539 and is characterized by the following structure:

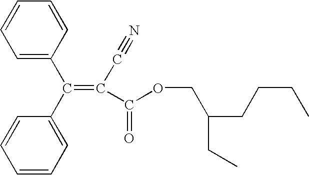

It can also be of considerable advantage to use polymer-bonded or polymeric UV filter substances in the preparations according to the present invention, in particular those described in WO-A-92/20690.

The list of specified UV filters which can be used in the sense of the present invention is not of course intended to be limiting.

The preparations according to the invention advantageously contain the substances which absorb UV radiation in the UV-A and/or UV-B region in a total amount of, for example, 0.1% by weight to 30% by weight, preferably 0.5 to 20% by weight, in particular 1.0 to 15.0% by weight, in each case based on the total weight of the preparations, in order to provide cosmetic preparations which protect the hair or the skin from the entire range of ultraviolet radiation. They can also be used as sunscreens for the hair or the skin.

The preparations present as emulsions according to the invention comprise one or more emulsifiers. These emulsifiers can advantageously be chosen from the group of nonionic, anionic, cationic or amphoteric emulsifiers.

The nonionic emulsifiers include

a) Partial fatty acid esters and fatty acid esters of polyhydric alcohols and ethoxylated derivatives thereof (e.g. glyceryl monostearates, sorbitan stearates, glyceryl stearyl citrates, sucrose stearates)

b) ethoxylated fatty alcohols and fatty acids

c) ethoxylated fatty amines, fatty acid amides, fatty acid alkanolamides

d) alkylphenol polyglycol ethers (e.g. Triton X)

The anionic emulsifiers include

a) soaps (e.g. sodium stearate)

b) fatty alcohol sulfates

c) mono-, di- and trialkylphosphoric esters and ethoxylates thereof

The cationic emulsifiers include

a) quaternary ammonium compounds with a long-chain aliphatic radical, e.g. distearyldimonium chloride

The amphoteric emulsifiers include

a) alkylamininoalkanecarboxylic acids

b) betaines, sulfobetaines

c) imidazoline derivatives

In addition, there are naturally occurring emulsifiers, which include beeswax, wool wax, lecithin and sterols.

O/W emulsifiers can be advantageously chosen, for example, from the group of polyethoxylated or polypropoxylated or polyethoxylated and polypropoxylated products, e.g.:

fatty alcohol ethoxylates

ethoxylated wool wax alcohols,

polyethylene glycol ethers of the general formula R-O-(-CH₂-CH₂-O-)ₙ-R′,

fatty acid ethoxylates of the general formula R-COO-(-CH₂-CH₂-O-)ₙ-H,

etherified fatty acid ethoxylates of the general formula R-COO-(-CH₂-CH₂-O-)ₙ-R′,

esterified fatty acid ethoxylates of the general formula R-COO-(-CH₂-CH₂-O-)ₙ-C(O)-R′,

polyethylene glycol glycerol fatty acid esters,

ethoxylated sorbitan esters,

cholesterol ethoxylates,

ethoxylated triglycerides,

alkyl ether carboxylic acids of the general formula R-O-(-CH₂-CH₂-O-)n-CH₂-COOH where n is a number from 5 to 30,

polyoxyethylene sorbitol fatty acid esters,

alkyl ether sulfates of the general formula R-O-(-CH₂-CH₂-O-)ₙ-SO₃-H,

fatty alcohol propoxylates of the general formula R-O-(-CH₂-CH(CH₃)-O-)ₙ-H,

olypropylene glycol ethers of the general formula R-O-(-CH₂-CH(CH₃)-O-)ₙ-R′,

propoxylated wool wax alcohols,

etherified fatty acid propoxylates R-COO-(-CH₂-CH(CH₃)-O-)ₙ-R′,

esterified fatty acid propoxylates of the general formula R-COO-(-CH₂-CH(CH₃)-O-)ₙ-C(O)-R′,

fatty acid propoxylates of the general formula R-COO-(-CH₂-CH(CH₃)-O-)ₙ-H,

polypropylene glycol glycerol fatty acid esters,

propoxylated sorbitan sters,

cholesterol propoxylates,

propoxylated triglycerides,

alkyl ether carboxylic acids of the general formula R-O-(-CH₂-CH(CH₃)O-)ₙ-CH₂-COOH,

alkyl ether sulfates or the parent acids of these sulfates of the general formula R-O-(-CH₂-CH(CH₃)-O-)ₙ-SO₃-H,

fatty alcohol ethoxylates/propoxylates of the general formula R-O-Xₙ-Yₘ-H,

polypropylene glycol ethers of the general formula R-O-Xₙ-Yₘ-R′,

etherified fatty acid propoxylates of the general formula R-COO-Xₙ-Yₘ-R′, and

fatty acid ethoxylates/propoxylates of the general formula R-COO-Xₙ-Yₘ-H.

According to the invention, particularly advantageous polyethoxylated or polypropoxylated or polyethoxylated and polypropoxylated ONV emulsifiers used are those chosen from the group of substances having HLB values of 11-18, very particularly advantageously having HLB values of 14.5-15.5, provided the ONV emulsifiers have saturated radicals R and R′. If the O/W emulsifiers have unsaturated radicals R and/or R′, or isoalkyl derivatives are present, then the preferred HLB value of such emulsifiers can also be lower or higher.

It is advantageous to choose the fatty alcohol ethoxylates from the group of ethoxylated stearyl alcohols, cetyl alcohols, cetylstearyl alcohols (cetearyl alcohols). Particular preference is given to: polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20), polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15), polyethylene glycol(12) lauryl ether (laureth-12), polyethylene glycol(12) isolauryl ether (isolaureth-12), polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol(19) cetylstearyl ether (ceteareth-19), and polyethylene glycol(20) cetylstearyl ether (ceteareth-20).

It is also advantageous to choose the fatty acid ethoxylates from the following group: polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, poly-ethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, and polyethylene glycol(20) oleate.

The ethoxylated alkyl ether carboxylic acid or salt thereof which can be used is advantageously sodium laureth-11 carboxylate.

Sodium laureth 1-4 sulfate can be used advantageously as alkyl ether sulfate.

An advantageous ethoxylated cholesterol derivative which can be used is polyethylene glycol(30) cholesteryl ether. Polyethylene glycol(25) soyasterol has also proven successful.

Ethoxylated triglycerides which can be advantageously used are polyethylene glycol(60) Evening Primrose glycerides.

It is also advantageous to choose the polyethylene glycol glycerol fatty acid esters from the group polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate, polyethylene glycol(20) glyceryl oleate, polyethylene glycol(20) glyceryl isostearate, polyethylene glycol(18) glyceryl oleate/cocoate.

It is likewise favorable to choose the sorbitan esters from the group polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

Advantageous W/O emulsifiers which can be used are: fatty alcohols having 8 to 30 carbon atoms, monoglycerol esters of saturated or unsaturated, branched or unbranched alkanecarboxylic acids having a chain length of from 8 to 24, in particular 12-18, carbon atoms, diglycerol esters of saturated or unsaturated, branched or unbranched alkanecarboxylic acids having a chain length of from 8 to 24, in particular 12-18, carbon atoms, monoglycerol ethers of saturated or unsaturated, branched or unbranched alcohols having a chain length of from 8 to 24, in particular 12-18, carbon atoms, diglycerol ethers of saturated or unsaturated, branched or unbranched alcohols having a chain length of from 8 to 24, in particular 12-18, carbon atoms, propylene glycol esters of saturated or unsaturated, branched or unbranched alkanecarboxylic acids having a chain length of from 8 to 24, in particular 12-18, carbon atoms, and sorbitan esters of saturated and/or unsaturated, branched or unbranched alkanecarboxylic acids having a chain length of from 8 to 24, in particular 12-18, carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprate, glyceryl monocaprylate.

The emulsions according to the invention can also advantageously comprise dyes and/or color pigments. The dyes and pigments can be chosen from the corresponding positive list of the Cosmetics Directive or the EC list of cosmetic colorants. In most cases they are identical to the dyes approved for foods. Advantageous color pigments are, for example, titanium dioxide, mica, iron oxides (e.g. Fe₂O₃, Fe₃O₄, FeO(OH)) and/or zinc oxide. Advantageous dyes are, for example, carmine, Berlin blue, chrome oxide green, ultramarine blue and/or manganese violet. It is particularly advantageous to choose the dyes and/or color pigments from the following list. The Colour Index Numbers (CIN) are taken from the Rowe Colour Index, 3^{rd} Edition, Society of Dyers and Colourists, Bradford, England, 1971.

| Chemical or other name | CIN | Color |
|---|---|---|
| Pigment Green | 10006 | green |
| Acid Green 1 | 10020 | green |
| 2,4-dinitrohydroxynaphthalene-7-sulphonic acid | 10316 | yellow |
| Pigment Yellow 1 | 11680 | yellow |
| Pigment Yellow 3 | 11710 | yellow |
| Pigment Orange 1 | 11725 | orange |
| 2,4-dihydroxyazobenzene | 11920 | orange |
| Solvent Red 3 | 12010 | red |
| 1-(2′-Chlor-4′-nitro-1′-phenylazo)-2-hydroxynaphthalene | 12085 | red |
| Pigment Red 3 | 12120 | red |
| Ceres Red, Sudan Red; Fat red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfodiethylamido-1′-phenylazo)-3-hydroxy- | 12490 | red |
| 5″-chloro-2″,4″-dimethoxy-2-naphthanilde | | |
| Disperse Yellow 16 | 12700 | yellow |
| 1-(4-Sulfo-1-phenylazo)-4-aminobenzene-5-sulfonic acid | 13015 | yellow |
| 2,4-Dihydroxyazobenzene-4′-sulfonic acid | 14270 | orange |
| 2-(2,4-Dimethylphenylazo-5-sulfonic acid)-1-hydroxynaphthalene- | 14700 | red |
| 4-sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfo-4-chloro-5-carboxy-1-phenylazo)-2- | 15525 | red |
| hydroxynaphthalene | | |
| 1-(3-Methylphenylazo-4-sulfo)-2-hydroxynaphthalene | 15580 | red |
| 1-(4′,(8′)-Sulfonaphthylazo)-2-hydroxynaphthalene | 15620 | red |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2-naphthylcarboxylic acid | 15850 | red |
| 1-(2-Sulfo-4-methyl-5-chloro-1-phenylazo)-2- | 15865 | red |
| hydroxynaphthalene-3- | | |
| 1-(2-Sulfo-1-naphthylazo)-2-hydroxynaphthalene-3-carboxylic | 15880 | red |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic acid | 16185 | red |
| Acid Orange 10 | 16230 | orange |
| 1-(4-Sulfo-1-naphthylazo)-2-naphthol-6,8-disulfonic acid | 16255 | red |
| 1-(4-Sulfo-1-naphthylazo)-2-naphthol-3,6,8-trisulfonic acid | 16290 | red |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hydroxy- | 19140 | yellow |
| Pigment Yellow 16 | 20040 | yellow |
| 2,6-(4′-Sulfo-2″,4″-dimethyl)-bisphenylazo)1,3-dihydroxybenzene | 20170 | orange |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]-1-hydroxy- | 27755 | black |
| 7-aminonaphthalene-3,6-disulfonic acid | | |
| 4′-[(4″-Sulfo-1″-phenylazo)-7′-sulfo-1′-naphthylazo]-1-hydoxy-8- | 28440 | black |
| acetylaminonaphthalene-3,5-disulfonic acid | | |
| Direct Orange 34, 39, 44, 46, 60 | 40215 | orange |
| Food Yellow | 40800 | orange |
| trans-β-Apo-8′-carotinaldehyde (C₃₀) | 40820 | orange |
| trans-Apo-8′-carotinic acid (C₃₀)-ethyl ester | 40825 | orange |
| Canthaxanthin | 40850 | orange |
| Acid Blue 1 | 42045 | blue |
| 2,4-Disulfo-5-hydroxy-4′-4″-bis-(diethylamino)triphenylcarbinol | 42051 | blue |
| 4-[(-4-N-Ethyl-p-sulfobenzylamino)-phenyl-(4-hydroxy-2- | 42053 | green |
| sulfophenyl)(methylene)-1-(N-ethyl, N-p-sulfobenzyl)-2,5- | | |
| cyclohexadienimine] | | |
| Acid Blue 7 | 42080 | blue |
| (N-Ethyl-p-sulfobenzylamino)-phenyl-(2-sulfophenyl)methylene- | 42090 | blue |
| (N-ethyl-N-p-sulfobenzyl)Δ^{2,5}-cyclohexadienimine | | |
| Acid Green 9 | 42100 | green |
| Diethyldisulfobenzyldi-4-amino-2-chloro-di-2-methyl-fuchsonimmonium | 42170 | green |
| Basic Violet 14 | 42510 | violet |
| Basic Violet 2 | 42520 | violet |
| 2′-Methyl-4′-(N-ethyl-N-m-sulfobenzyl)-amino-4″-(N-diethyl)- | 42735 | blue |
| amino-2-methyl-N-ethylN-m-sulfobenzylfuchsonimmonium | | |
| 4′-(N-Dimethyl)-amino-4″-(N-phenyl)-aminonaphtho-N-dimethylfuchsonimmonium | 44045 | blue |
| 2-Hydroxy-3,6-disulfo-4,4′-bisdimethylaminonaptha- | 44090 | green |
| fuchsonimmonium | | |
| Acid Red 52 | 45100 | red |
| 3-(2′-Methylphenylamino)-6-(2′-methyl-4′-sulfophenylamino)-9- | 45190 | violet |
| (2″-carboxyphenyl) xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminum complex | 58000 | red |
| 3-oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)-anthraquinone | 60725 | violet |
| Acid Violet 23 | 60730 | violet |
| 1,4-Di(4′-methylphenylamino)anthraquinone | 61565 | green |
| 1,4-Bis-(o-sulfo-p-toluidino)anthraquinone | 61570 | green |
| Acid Blue 80 | 61585 | blue |
| Acid Blue 62 | 62045 | blue |
| N,N′-Dihydro-1,2,1′,2′-anthraquinone azine | 69800 | blue |
| Vat Blue 6; Pigment Blue 64 | 69825 | blue |
| Vat Orange 7 | 71105 | orange |
| Indigo | 73000 | blue |
| Indigo-disulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanine | 74260 | green |
| Natural Yellow 6,19; Natural Red 1 | 75100 | yellow |
| Bixin, Norbixin | 75120 | orange |
| Lycopene | 75125 | yellow |
| trans-alpha-, beta- and gamma-carotene | 75130 | orange |
| Keto-and/or hydroxyl derivatives of carotene | 75135 | yellow |
| Guanine or pearlizing agent | 75170 | white |
| 1,7-Bis-(4-hydroxy-3-methoxyphenyl)1,6-heptadien-3,5-dione | 75300 | yellow |
| complex salt (Na, Al, Ca) of carminic acid | 75470 | red |
| Chlorophyll a and b; copper compounds of chlorophylls and | 75810 | green |
| Aluminum | 77000 | white |
| Hydrated alumina | 77002 | white |
| Hydrous aluminum silicates | 77004 | white |
| Ultramarine | 77007 | blue |
| Pigment Red 101 and 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268:1 | black |
| Chromium oxide | 77288 | green |
| Chromium oxide, hydrous | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Iron oxide, hydrated | 77492 | yellow |
| Iron oxide | 77499 | black |
| Mixtures of iron (II) and iron (III) hexacyanoferrate | 77510 | blue |
| Pigment White 18 | 77713 | white |
| Manganese animonium diphosphate | 77742 | violet |
| Manganese phosphate, Mn₃(PO₄)₂ 7 H20 | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)-isoalloxazine, lactoflavine | | yellow |
| Sugar coloring | | brown |
| Capsanthin, capsorubin | | orange |
| Betanin | | red |
| Benzopyrylium salts, Anthocyans | | red |
| Aluminum, zinc, magnesium and calcium stearate | | white |
| Bromothymol blue | | blue |
| Bromocresol green | | green |
| Acid Red 195 | | red |

It can also be favorable to choose one or more substances from the following group as the dye: 2,4-dihydroxyazobenzene, 1-(2′-chloro4′-nitro-1′-phenylazo)-2-hydroxynaphthalene, Ceres Red, 2-(4-sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 4-(4-sulfo-1-phenylazo)-1-(4-sulfophenyl)-5-hydroxypyrazolone-3-carboxylic acid, aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromofluorescein and its aluminum salt, aluminum salt of 2,4,5,7-tetraiodofluorescein, aluminum salt of quinophthalone disulfonic acid, aluminum salt of indigo disulfonic acid, red and black iron oxide (CIN: 77 491 (red) and 77 499 (black)), iron oxide hydrate (CIN: 77 492), manganese ammonium diphosphate and titanium dioxide.

Also advantageous are oil-soluble natural dyes, such as, for example, paprika extracts, carotene or cochenille.

Also advantageous in the sense of the present invention are gel creams with a content of pearlescent pigments. Preference is given in particular to the types of pearlescent pigments listed below:

Natural pearlescent pigments, such as, for example

“pearl essence” (guanine/hypoxanthin mixed crystals from fish scales) and

“mother of pearl” (ground mussel shells)

Monocrystalline pearlescent pigments, such as, for example, bismuth oxychloride (BiOCl)

Layer substrate pigments: e.g. mica/metal oxide

Bases for pearlescent pigments are, for example, pulverulent pigments or castor oil dispersions of bismuth oxychloride and/or titanium dioxide, and bismuth oxichloride and/or titanium dioxide on mica. The luster pigment listed under CIN 77163, for example, is particularly advantageous.

Also advantageous are, for example, the following types of pearlescent pigment based on mica/metal oxide:

| Group | Coating/Layer | Color |
|---|---|---|
| Silver-white pearlescent | TiO₂: 40-60 nm | silver |
| Interference pigments | TiO₂: 60-80 nm | yellow |
| | TiO₂: 80-100 nm | red |
| | TiO₂: 100-140 nm | blue |
| | TiO₂: 120-160 nm | green |
| Color luster pigments | Fe₂O₃ | bronze |
| | Fe₂O₃ | Copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Berlin blue | Deep blue |
| | TiO₂/Carmine | red |

Particular preference is given, for example, to the pearlescent pigments obtainable from Merck under the trade names Timiron, Colorona or Dichrona.

The list of given pearlescent pigments is not of course intended to be limiting. Pearlescent pigments which are advantageous for the purposes of the present invention are obtainable by numerous methods known per se. For example, other substrates apart from mica can be coated with further metal oxides, for example silica and suchlike. SiO₂ particles coated with, for example, TiO₂ and Fe₂O₃ (“ronaspheres”), which are marketed by Merck and are particularly suitable for the visual diminution of fine wrinkles.

It may moreover be advantageous to dispense with a substrate, such as mica, entirely. Pearlescent pigments which are prepared using SiO₂ are particularly preferred. Such pigments, which can also additionally have gonichromatic effects, are obtainable, e.g., from BASF under the trade name Sicopearl Fantastico.

Pigments from Engelhard/Mearl which are based on calcium sodium borosilicate and are coated with titanium dioxide can, furthermore, advantageously be employed. These are obtainable under the name Reflecks. Due to their particle size of 40-180 μm, they have a glitter effect, in addition to the color.

In addition, also particularly advantageous are effect pigments which are obtainable under the trade name Metasomes Standard/Glitter in various colors (yellow, red, green, blue) from Flora Tech. The glitter particles are present here in mixtures with various auxiliaries and dyes (such as, for example, the dyes with the Colour Index (CI) numbers 19140, 77007, 77289, 77491).

The dyes and pigments may be present either individually or in a mixture, and can be mutually coated with one another, different coating thicknesses generally giving rise to different color effects. The total amount of dyes and color-imparting pigments is advantageously chosen from the range from, for example, 0.1% by weight to 30% by weight, preferably from 0.5 to 20% by weight, in particular from 1.0 to 15% by weight, in each case based on the total weight of the preparations.

Preparations according to the invention can advantageously also comprise powders. Powders are pulverulent preparations composed of one or more powder bases which have a greater or lesser finely divided nature and to which, depending on their intended use, one or more active ingredients, preservatives, perfume oils, dyes, etc can be added.

The FDA's OTC Miscellaneous External Panel has stipulated the following definition for powders: “A homogeneous dispersion of finely dispersed, relatively dry finely divided material which consists of one or more substances” (FDC Reports [Pink Sheet] 41, No. 33, T&G-4 [Aug. 13, 1979]).

The composition of a powder depends largely on the objectives which it has to fulfil. Powders can, however, also be diluents for medicaments, e.g. antibiotics, sulphonamides, etc. Liquid powders are mostly high-viscosity preparations (lotions) consisting of talc, zinc oxide and/or titanium dioxide, glycerol and water. Compact powders are powder bases briquetted by high pressure or caked together by adding calcium sulphate (gypsum).

Additionally, powders are also provided and used in aerosol form after it was possible to develop valves which largely exclude the possibility of the valve execution operations being obstructed.

The sedimentation of the incorporated powder particles, which is always a risk, can likewise be prevented by incorporating suitable suspending agents and/or suspension auxiliaries into the formulation, for example alkali metal, ammonium or amine salts of a dialkyl sulphosuccinate with alkyl groups of 4-12 carbon atoms, e.g. sodium dioctyl sulphosuccinate (typically about 0.002-0.015% by weight), or an alkylbenzenesulphonic acid with alkyl groups of 8-14 carbon atoms, e.g. sodium dodecylbenzenesulphonate.

Preparations according to the invention can also advantageously contain thickeners. Suitable thickeners are:

Homopolymers of acrylic acid with a molecular weight of 2,000,000 to 6,000,000, such as the trade product Carbopole. Additional thickeners are sold under the names Carbopol 940, Carbopol EDTA 2001 or Modarez V 600 PX. Polymers comprising acrylic acid and acrylamide (sodium salt) with a molecular weight of 2,000,000 to 6,000,000, such as Hostacerin PN 73 or the sclerotium gum sold under the name Amigel.

Also suitable are copolymers of acrylic acid or methacrylic acid, such as Carbopol 1342 or Permulen TRI.

Further thickener types are polyglycols, cellulose derivatives, in particular hydroxyalkyl celluloses as well as alginates, carageenan and inorganic thickeners, such as natural or synthetic bentonites.

The antioxidants are advantageously selected from the group consisting of amino acids (e.g. glycine, lysine, histidine, tyrosine, tryptophan) and their derivatives as a salt, ester, ether, sugar, nucleotide, nucleoside, peptide and lipid compound), imidazoles (e.g. urocanic acid) and their derivatives (as a salt, ester, ether, sugar, nucleotide, nucleoside, peptide and/or lipid compound), peptides, such as D,L-carnosine, D-carnosine, L-carnosine, anserine and their derivatives (as a salt, ester, ether, sugar, thiol, nucleotide, nucleoside, peptide and lipid compound), carotenoids, carotenes (e.g. α-carotene, β-carotene, ψ-lycopene, phytonene) and their derivatives (as a salt, ester, ether, sugar, nucleotide, nucleoside, peptide and/or lipid compound), chlorogenic acid and derivatives thereof (as a salt, ester, ether, sugar, thiol, nucleotide, nucleoside, peptide and/or lipid compound), aurothioglucose, propylthiouracil and other thiols (e.g. thioredoxin, lipoic acid, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, distearyl thiodipropionate, thiodipropionic acid and their derivatives (as a salt, ester, ether, sugar, thiol, nucleotide, nucleoside, peptide and/or lipid compound) and sulfoximine compounds (e.g. homocysteine sulfoximine, buthionine sulfones, penta-, hexa-, heptathionine sulfoximine) in very low tolerated doses (e.g. pmol to Nmol/kg). Also (metal) chelating agents (e.g. apoferritin, desferal, lactoferrin, α-hydroxy fatty acids, palmitic acid, phytic acid) and their derivatives (as a salt, ester, ether, sugar, thiol, nucleotide, nucleoside, peptide and/or lipid compound), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. y-linolenic acid, linoleic acid, oleic acid), folic acid and its derivatives, furfurylidenesorbitol and its derivatives, ubiquinone, ubiquinol, plastoquinone and their derivatives (as a salt, ester, ether, sugar, thiol, nucleotide, nucleoside, peptide and lipid compound), vitamin C and derivatives (e.g. ascorbyl palmitate, Mg ascorbyl phosphate, ascorbyl acetate), tocopherols and derivatives (e.g. vitamin E acetate), as well as phenolic compounds and plant extracts containing this, such as e.g. flavonoids (e.g. glycosylrutin, ferulic acid, caffeic acid), furfurylideneglucitol, butylhydroxytoluene. butylhydroxyanisole, nordihydrogualacic acid, nordihydrogualaretic acid, trihydroxybutyrophenone and their derivatives (as a salt, ester, ether, sugar, nucleotide, nucleoside, peptide and lipid compound), uric acid and its derivatives, mannose and its derivatives (as a salt, ester, ether, sugar, thiol, nucleotide, nucleoside, peptide and lipid compound), zinc and its derivatives (e.g. ZnO, ZnSO₄), selenium and its derivatives (e.g. selenomethionine, ebselen), stilbenes and their derivatives (e.g. stilbene oxide, trans-stilbene oxide) and the derivatives (as a salt, ester, ether, sugar, thiol, nucleotide, nucleoside, peptide and/or lipid compound) of the said active ingredients which are suitable according to the invention.

It is preferable for the purpose of the invention to add complexing agents containing such active substance combinations to the active substance combinations or cosmetic or dermatological preparations according to the invention.

Complexing agents are additives of cosmetology or medical Galenism known per se. The complexing of interfering metals such as Mn, Fe, Cu and other such metals enables, for example, undesired chemical reactions in cosmetic or dermatological preparations to be prevented.

Complexing agents, in particular chelating agents, form complexes with metal atoms which represent metallacycles if one or more polybasic complex forms, that is chelating agents, are present. Chelates represents compounds in which an individual ligand occupies more than one coordination site in a central atom. In this case, normally linear compounds are closed to form rings through complex formation via a metal atom or ion. The number of bound ligands depends on the coordination number of the central metal. Chelate formation requires that the compound reacting with the metal contains two or more atom groups which act as electron donors.

The complexing agent(s) can advantageously be selected from the group of standard compounds, whereby at least one substance is preferred from the group consisting of tartaric acid and its anions, citric acid and its anions, aminopolycarboxylic acids and their anions (such as, for example, ethylenediaminetetraacetic acid (EDTA) and its anions, nitrilotriacetic (NTA) and its anions, hydroxyethylenediaminotriacetic acid (HOEDTA) and its anions, diethyleneaminopentaacetic acid (DPTA) and its anions, trans-1,2-diaminocyclohexanetetraacetic acid (CDTA) and its anions).

According to the invention, the complexing agent(s) is/are advantageously present in cosmetic or dermatological preparations preferably to 0.001% by weight to 10% by weight, preferably to 0.01% by weight to 5% by weight, particularly preferred to 0.05-2.0% by weight, based on the total weight of the preparations.

Dimethicone copolyol (and) polyglyceryl-4-isostearate (and) hexyl laurate. It can be advantageous, even if such is not essential, if the preparations according to the invention contain preservatives.

Advantageous preservatives for the purposes of the present invention are, for example, formaldehyde donors (such as DMDM hydantoin, which is available, for example, under the trade name Glydant™ from Lonza), iodopropyl butylcarbamates (e.g. those available under the trade names Glycacil-L, Glycacil-S from the company Lonza and/or Dekaben LMB from Jan Dekker), parabens (i.e. p-hydroxybenzoic alkyl esters, such as methyl-, ethyl-, propyl- and/or butylparaben), phenoxyethanol, ethanol, benzoic acid and the like.

Usually, according to the invention, the preservative system also advantageously comprises preservative assistants, such as, for example, octoxyglycerol, glycine soya etc. This list of advantageous preservatives is in no way intended to be restrictive. Rather, all preservatives approved for cosmetics and foodstuffs are advantageous for the purpose of the present invention.

It is possible in all of this in the individual case that the aforementioned concentration data are slightly exceeded or fallen short of, but nevertheless preparations according to the invention are obtained. In view of the widespread variety of suitable components of such preparations, this is not unexpected for the person skilled in the art, and the lafter will therefore know that in the case of such an exceeding or falling short, the base of the invention is not left.

### EXAMPLES

The following examples are intended to illustrate the present invention without restricting it. The numerical values in the examples denote percentages by weight, based on the total weight of the respective preparations.

### Example 1

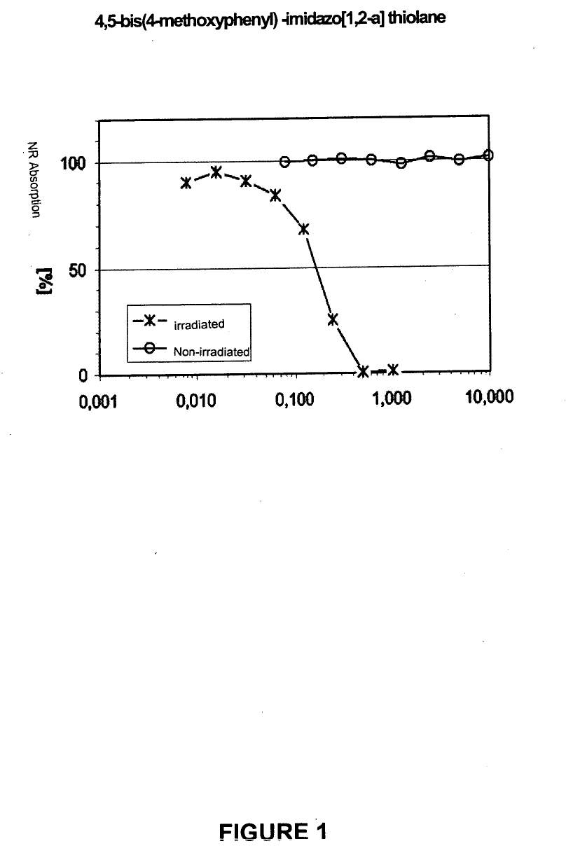

The photocytotoxicity of 4,5-bis(4-methoxyphenyl)-imidazo[1,2-a]thiolane-1-oxide and 4,5-bis(4-methoxyphenyl)-imidazo[1,2-a]thiolane-1-dioxide in comparison to 4,5-bis(4-methoxyphenyl)-imidazo[1,2-a]thiolane according to standard protocol “3T3 NRU Phototoxicity Assay” of the COLIPA Validation Ring Study of 1997 is shown in FIG. 1.

In the test method used, the change to the neutral red absorption of 3T3 cells is measured by UV irradiation. Only vital cells absorb the dye, a reduced absorption thus indicates a toxicity of the test substance added. The so-called NR50 value is typically determined for evaluation, i.e. those concentrations of the test substance for which the neutral red absorption has fallen to 50% of the check value. In order to determine the phototoxicity, this value is determined for non-irradiated and irradiated cultures and put in a ratio. The lower the quotient, the lower the phototoxicity (see Table 1).

TABLE 1

| | NR50 | NR50 | Quotient |
|---|---|---|---|
| | non-irradiated | irradiated | non-irradiated/ |
| | mg/l | mg/l | irradiated PIF |
| 4,5-bis(4-methoxyphenyl)- | >10 | 0.177 | >56 |
| imidazo[1,2-a]thiolane | | | |
| 4,5-bis(4-methoxyphenyl)- | >100 | >42.3 | >2.4 |
| imidazo[1,2-a]thiolane- | | | |
| 1-oxide | | | |
| 4,5-bis(4-methoxyphenyl)- | >50 | 7.77 | >6.4 |
| imidazo[1,2-a]thiolane- | | | |
| 1-dioxide | | | |

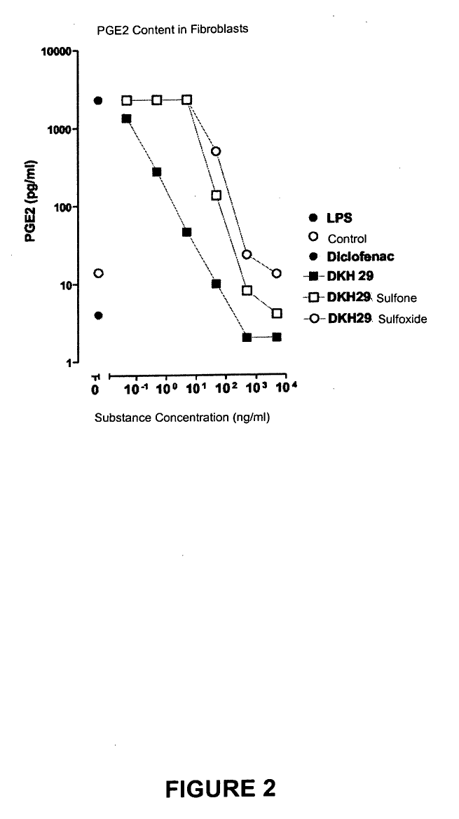

The influence of 4,5-bis(4-methoxyphenyl)-imidazo[1,2-a]thiolane-1-oxide and 4,5-bis(4-Methoxyphenyl)-imidazo[1,2-a]thiolane-1-dioxide in comparison to 4,5-bis(4-methoxyphenyl)-imidazo[1,2-a]thiolane on eicosanoid production is illustrated in FIG. 2. For the samples in FIG. 2, human fibroblasts were stimulated with LPS and 24 hours later the supernatant PGE₂ content was determined. Either Diclofenac with standard effects or dilutions of DKH derivatives were added to the LPS-stimulated cultures. Non-stimulated cultures and cultures only stimulated with LPS were measured as a control test.

Human fibroblasts in culture were induced to release prostaglandin E₂ (PGE₂) by means of lipopolysaccharide (LPS). Inhibition of this PGE₂ production by imidazoles was then determined in the test. Good inhibitors exhibit effectiveness in the micromolar range and below. FIG. 2 reveals that although the capacity for inhibition of cyclooxygenases is reduced through the oxidation of sulfur to sulfoxide or sulfone, sufficient inhibition in the concentration range below one microgram per millimeter is still nevertheless attained.

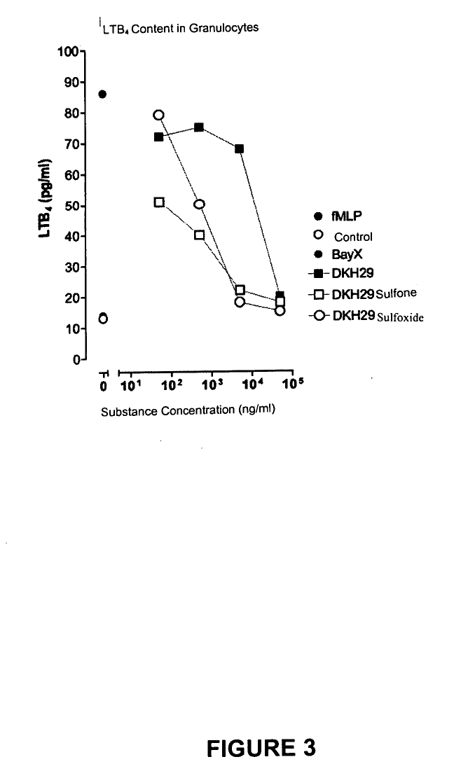

A second test method was used to determine the influence of imidazoles on the release of LTB₄ by stimulated granulocytes. Human neutrophile granulocytes from the peripheral blood were isolated, cultured and stimulated for LTB4 production with formylated methionyl-leucyl-phenylalanine (fMLP). Specifically, human neutrophile granulocytes were stimulated with fMLP and 2 hours later the supernatant LTB₄ content was determined. Either BayX1005 with standard effects or dilutions of DKH derivatives were added to the fMLP-stimulated cultures. Non-stimulated cultures and cultures only stimulated with fMLP were measured as a control test. The results are provided in FIG. 3 and revealed that both the sulfoxide and sulfone enabled improved inhibition of LTB4 release.

### Formulation Examples

### PIT Emulsions

The fat and water phases were heated separately to 80° C. The fat phase is presented. At 80° C. the perfume is added, then the water phase is added. The emulsion is cooled to room temperature by stirring. A homogenization is not required on account of the spontaneous formation of the emulsion.

| | 1 | 2 | 3 | 4 | 5 |
|---|---|---|---|---|---|
| Glycerol monostearate, self-emulsifying | 0.50 | | 3.00 | 2.00 | 4.00 |
| Polyoxyethylene(12) cetylstearyl ether | | 5.00 | | 1.00 | 1.50 |
| Polyoxyethylene(20) cetylstearyl ether | | | | 2.00 | |
| Polyoxyethylene(30) cetylstearyl ether | 5.00 | | 1.00 | | |
| Stearyl alcohol | | | 3.00 | | 0.50 |
| Cetyl alcohol | 2.50 | 1.00 | | 1.50 | |
| 2-Ethylhexyl methoxycinnamate | | | | 5.00 | 8.00 |
| 2,4-Bis-(4-(2-ethylhexyloxy-)2-hydroxyl)- | | 1.50 | | 2.00 | 2.50 |
| phenyl)-6-(4-methoxyphenyl)-(1,3,5)-triazine | | | | | |
| 1-(4-tert-Butylphenyl)-3-(4-methoxyphenyl)- | | | 2.00 | | |
| 1,3-propandione | | | | | |
| Diethylhexylbutamidotriazone | 1.00 | 2.00 | | 2.00 | |
| Ethylhexyltriazone | 4.00 | | 3.00 | 4.00 | |
| 4-Methylbenzylidenecamphor | | 4.00 | | | 2.00 |
| Octocrylene | | 4.00 | | | 2.50 |
| Phenylene-1,4-bis-(monosodium, 2- | | | 0.50 | | 1.50 |
| benzimidazyl-5,7-disulfonic acid | | | | | |
| Phenylbenzimidazolesulfonic acid | 0.50 | | | 3.00 | |
| C12-15 Alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol dicaprylate/dicaprate | 5.00 | | | 6.00 | |
| Dicaprylyl carbonate | | | 6.00 | | 2.00 |
| Dimethicone polydimethylsiloxane | | 0.50 | 1.00 | | |
| Phenylmethylpolysiloxane | 2.00 | | | 0.50 | 0.50 |
| Shea butter | | 2.00 | | | 0.50 |
| PVP hexadecane copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| 4,5-bis(4-methoxyphenyl)-imidazo[1,2- | 0.30 | 0.10 | 0.60 | 0.20 | 0.30 |
| a]thiolane-1-oxide | | | | | |
| Alpha-Glucosylrutin | 0.10 | | 0.20 | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

O/W cream examples

| | 1 | 2 | 3 | 4 | 5 |
|---|---|---|---|---|---|
| Glyceryl stearate citrate | | | 2.00 | | 2.00 |
| Glyceryl stearate, self-emulsifying | 4.00 | 3.00 | | | |
| PEG-40 stearate | 1.00 | | | | |
| Polyglyceryl-3-methylglucose distearate | | | | 3.00 | |
| Sorbitan stearate | | | | | 2.00 |
| Stearic acid | | 1.00 | | | |
| Polyoxyethylene(20) cetylstearyl ether | | | | | |
| Stearyl alcohol | | | 5.00 | | |
| Cetyl alcohol | 3.00 | 2.00 | | 3.00 | |
| Cetylstearylal kohol | | | | | 2.00 |
| C12-15 alkyl benzoate | | | | | |
| Caprylic/Capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| 4-Methylbenzylidenecamphor | | | 1.00 | | |
| 1-(4-tert-Butylphenyl)-3-(4-methoxyphenyl)-1,3- | | | 0.50 | | |
| Propandione | | | | | |
| 4,5-bis(4-methoxyphenyl)-imidazo[1,2- | 0.20 | 0.50 | 0.10 | 1.00 | 0.30 |
| a]thiolane-1-oxide | | | | | |
| Tocopherol | 0.1 | | | | 0.20 |
| Biotin | | | 0.05 | | |
| Ethylenediaminetetraacetic acid trisodium | 0.1 | | 0.10 | 0.1 | |
| Preservative | q.s. | q.s. | q.s. | q.s. | q.s. |
| Xanthan gum | | | | | |
| Polyacrylic acid | 3.00 | 0.1 | | 0.1 | 0.1 |
| Sodium hydroxide solution 45% | q.s | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | ad 100 | ad 100 | ad 100 | ad 100 |

O/W cream examples

| | 6 | 7 | 8 | 9 | 10 |
|---|---|---|---|---|---|
| Glyceryl stearate citrate | | 2.00 | 2.00 | | |
| Glyceryl stearate, self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetylstearyl alcohol | | 3.00 | 1.00 | | 0.50 |
| C12-15 Alkyl benzoate | | 2.00 | 3.00 | | |
| Caprylic/Capric triglyceride | 2.00 | | | | |
| Octyldodecanol | 2.00 | 2.00 | | 4.00 | 6.00 |
| Dicaprylyl ether | | | | | |
| paraffinum liquidum | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| 4-Methylbenzylidencamphor | 1.00 | | | | 1.00 |
| 1-(4-tert-Butylphenyl)-3-(4-methoxyphenyl)-1,3- | 0.50 | | | | 0.50 |
| Propandione | | | | | |
| 4,5-bis(4-methoxyphenyl)-imidazo[1,2- | 0.20 | 0.70 | 0.25 | 1.00 | 0.40 |
| a]thiolane-1-dioxide | | | | | |
| Tocopherol | | | | | 0.05 |
| Ethylendiaminetetraacetic acid trisodium | | | 0.20 | | 0.20 |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| Sodium hydroxide solution 45% | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | ad 100 | ad 100 | ad 100 | ad 100 | ad 100 |

W/O emulsion examples

| | 1 | 2 | 3 | 4 | 5 |
|---|---|---|---|---|---|
| Cetyldimethicone copolyol | | 2.50 | | 4.00 | |
| Polyglyceryl-2-dipolyhydroxystearate | 5.00 | | | | 4.50 |
| PEG-30 dipolyhydroxystearate | | | 5.00 | | |
| 2-Ethylhexyl methoxycinnamate | | 8.00 | | 5.00 | 4.00 |
| 2,4-bis-(4-(2-ethylhexyloxy-)2-hydroxyl)-phenyl)- | 2.00 | 2.50 | | 2.00 | 2.50 |
| 6-(4-methoxyphenyl)-(1,3,5)-triazine | | | | | |
| 1-(4-tert-Butylphenyl)-3-(4-methoxyphenyl)-1,3- | | | 2.00 | 1.00 | |
| Propanedione | | | | | |
| Diethylhexylbutamidotriazone | 3.00 | 1.00 | | | 3.00 |
| Ethylhexyltriazone | | | 3.00 | 4.00 | |
| 4-Methylbenzylidene camphor | | 2.00 | | 4.00 | 2.00 |
| Octocrylene | 7.00 | 2.50 | 4.00 | | 2.50 |
| Diethylhexylbutamidotriazone | 1.00 | | | 2.00 | |
| Phenylene-1,4-bis-(monosodium, 2- | 1.00 | 2.00 | 0.50 | | |
| benzimidazyl-5,7-disulfonic acid) | | | | | |
| Phenylbenzimidazolesulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Paraffinum liquidum | | | 10.0 | | 8.00 |
| C12-15 Alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethylpolysiloxane | 2.00 | 25.00 | | | 2.00 |
| Shea butter | | | 3.00 | | |
| PVP hexadecane copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| 4,5-bis(4-methoxyphenyl)-imidazo[1,2- | 0.10 | 0.60 | 1.00 | 1.00 | 0.80 |
| a]thiolane-1-Oxide | | | | | |
| Preservative | q.s. | q.s. | q.s. | q.s. | q.s. |
| Ethanol | 3.00 | | 1.50 | | 1.00 |
| Perfume | q.s. | q.s. | q.s. | q.s. | q.s. |
| Water | ad 100 | ad 100 | ad 100 | ad 100 | ad 100 |

W/O emulsion examples

| | 6 | 7 |
|---|---|---|
| Polyglyceryl-2-dipolyhydroxystearate | 4.00 | 5.00 |
| PEG-30 dipolyhydroxystearate | | |
| Lanolin alcohol | 0.50 | 1.50 |
| Isohexadecane | 1.00 | 2.00 |
| Myristyl myristate | 0.50 | 1.50 |
| Vaseline | 1.00 | 2.00 |
| 1-(4-tert-Butylphenyl)-3-(4-methoxyphenyl)-1,3- | 0.50 | 1.50 |
| Propandione | | |
| 4-Methylbenzylidenecamphor | | 3.00 |
| Butylene glycol dicaprylate/dicaprate | 4.00 | 5.00 |
| Shea butter | | 0.50 |
| Butylene glycol | | 6.00 |
| Octoxyglycerol | | 3.00 |
| Glycerol | 5.00 | |
| Tocopherol acetate | 0.50 | 1.00 |
| 4,5-bis(4-methoxyphenyl)-imidazo[1,2- | 0.20 | 0.25 |
| a]thiolane-1-Oxide | | |
| Trisodium EDTA | 0.20 | 0.20 |
| Preservative | q.s. | q.s. |
| Ethanol | | 3.00 |
| Perfume | q.s. | q.s. |
| Water | ad 100 | ad 100 |

Hydrodispersion examples

| | 1 | 2 | 3 | 4 | 5 |
|---|---|---|---|---|---|
| Polyoxyethylene(20) cetylstearyl ether | 1.00 | | | 0.5 | |
| Cetyl alcohol | | | 1.00 | | |
| Sodium polyacrylate | | 0.20 | | 0.30 | |
| Acrylates/C10-30-alkyl acrylate crosspolymer | 0.50 | | 0.40 | 0.10 | 0.10 |
| Xanthan gum | | 0.30 | 0.15 | | 0.50 |
| 2-Ethylhexyl methoxycinnamate | | | | 5.00 | 8.00 |
| 2,4-bis-(4-(2-ethylhexyloxy-)2-hydroxyl)- | | 1.50 | | 2.00 | 2.50 |
| phenyl)-6-(4-methoxyphenyl)-(1,3,5)-triazine | | | | | |
| 1-(4-tert-Butylphenyl)-3-(4-methoxyphenyl)-1,3- | 1.00 | | 2.00 | | |
| Propandione | | | | | |
| Diethylhexylbutamidotriazone | | 2.00 | | 2.00 | 1.00 |
| Ethylhexyltriazone | 4.00 | | 3.00 | 4.00 | |
| 4-Methylbenzylidenecamphor | | 4.00 | | | 2.00 |
| Octocrylene | | 4.00 | 4.00 | | 2.50 |
| Phenylene-1,4-bis-(monosodium, 2- | 1.00 | | 0.50 | | 2.00 |
| benzimidazyl-5,7-disulfonic acid | | | | | |
| Phenylbenzimidazolesulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-15 Alkyl benzoate | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP hexadecane copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| 4,5-bis(4-methoxyphenyl)-imidazo[1,2- | 0.15 | 0.60 | 1.00 | 1.00 | 0.80 |
| a]thiolane-1-Oxide | | | | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s |
| Water | ad 100 | ad 100 | ad 100 | ad 100 | ad 100 |

Example (Gel Cream):

| | |
|---|---|
| Acrylate/C10-30 alkyl acrylate crosspolymer | 0.40 |
| Polyacrylic acid | 0.20 |
| Xanthan gum | 0.10 |
| Cetearyl alcohol | 3.00 |
| C12-15 alkyl benzoate | 4.00 |
| Caprylic/Capric triglyceride | 3.00 |
| Cyclic dimethylpolysiloxane | 5.00 |
| Dimeticone polydimethylsiloxane | 1.00 |
| 4,5-bis(4-methoxyphenyl)-imidazo[1,2- | 0.20 |
| a]thiolane-1-Oxide | |
| Glycerol | 3.00 |
| Sodium hydroxide | q.s. |
| Preservative | q.s. |
| Perfume | q.s. |
| Water | ad 100 |
| pH value adjusted to 6.0 | |

Example (W/O Cream)

| | |
|---|---|
| Polyglyceryl-3-diisostearate | 3.50 |
| Glycerol | 3.00 |
| Polyglyceryl-2-dipolyhydroxystearate | 3.50 |
| 4,5-bis(4-methoxyphenyl)-imidazo[1,2- | 0.50 |
| a]thiolane-1-Oxide | |
| Preservative | q.s. |
| Perfume | q.s. |
| Water | ad 100 |
| Magnesium sulfate | 0.6 |
| Isopropyl stearate | 2.0 |
| Caprylyl ether | 8.0 |
| Cetearyl isononanoate | 6.0 |

Example (W/O/W Cream):

| | |
|---|---|
| Glyceryl stearate | 3.00 |
| PEG-100 stearate | 0.75 |
| Behenyl alcohol | 2.00 |
| Caprylic/Capric triglyceride | 8.0 |
| Octyldodecanol | 5.00 |
| C12-15 Alkyl benzoate | 3.00 |
| 4,5-bis(4-methoxyphenyl)-imidazo[1,2- | 1.00 |
| a]thiolane-1-Oxide | |
| Magnesium sulfate (MgSO₄) | 0.80 |
| Ethylenediaminetetraacetic acid | 0.10 |
| Preservative | q.s. |
| Perfume | q.s. |
| Water | ad 100 |
| pH value adjusted to 6.0 | |

Example (OW Emulsion):

| | |
|---|---|
| Triceteareth-4-phosphate | 0.70 |
| Glyceryl lanolate | 1.50 |
| Cyclic dimethylpolysiloxane | 1.00 |
| Butylene glycol | 3.0 |
| Carbomer | 0.45 |
| Sodium hydroxide solution 45% | 0.30 |
| 4,5-bis(4-methoxyphenyl)-imidazo[1,2- | 0.50 |
| a]thiolane-1-Oxide | |
| Isopropyl palmitate | 0.50 |
| Ethylenediaminetetraacetic acid | 1.00 |
| Preservative | q.s. |
| Perfume | q.s. |
| Water | ad 100 |

1. A topical, anti-inflammatory cosmetic, dermatological, or medical preparation comprising at least one compound of the formula 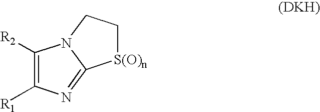
wherein
n is 1 or 2; and
R₁ and R₂, independently of each other, are selected from the group consisting of phenyl, 2-tolyl, 3-tolyl, 4-tolyl, 2-methoxyphenyl, 3-methoxyphenyl, and 4-methoxyphenyl.

2. The preparation as claimed in claim 1, wherein R₁ and R₂ are 4-methoxyphenyl.

3. The preparation as claimed in claim 1, wherein n=1.

4. The preparation as claimed in claim 1, wherein the at least one compound of the formula (DKH) is present in a concentration of from 0.0001 % to 10% by weight.

5. The preparation as claimed in claim 1, wherein the at least one compound of the formula (DKH) is present in a concentration of from 0.001 to 1% by weight.

6. The preparation as claimed in claim 1, wherein the at least one compound of the formula (DKH) is present in a concentration of from 0.01 to 0.1% by weight.

7. The preparation as claimed in claim 1, wherein the at least one compound of the formula (DKH) comprises 4,5-bis(4-methoxyphenyl)-imidazo[1,2a]thiolane-1-oxide and 4,5-bis(4-methoxyphenyl)-imidazo[1,2a]thiolane-1-dioxide.

8. The preparation as claimed in claim 1, further comprising at least one skin moisturizing agent.

9. The preparation as claimed in claim 8, wherein the at least one skin moisturizing agent is selected from the group consisting of glycerol, chitosan, Fucogel, propylene glycol, dipropylene glycol, butylene glycol, mannitol, lactic acid, salts of lactic acid, sodium pyrrolidonecarboxylic acid, salts of sodium pyrrolidonecarboxylic acid, hyaluronic acid, salts of hyaluronic acid, glycine, and urea.

10. The preparation as claimed in claim 8, wherein the at least one skin moisturizing agent is selected from the group consisting of glycerol, lactic acid, butylene glycol, urea, and hyaluronic acid.

11. The preparation as claimed in claim 8, wherein the at least one skin moisturizing agent is present in a concentration of 5-40% by weight, based on the total weight of the preparation.

12. The preparation as claimed in claim 1, further comprising at least one UV filter substance.

13. The preparation as claimed in claim 12, wherein the at least one UV filter substance is present in a total concentration of 1-15% by weight, based on the total weight of the preparation.

14. The preparation as claimed in claim 1, further comprising at least one substance selected from the group consisting of pigments, dyes, powders, and thickeners.

15. The preparation as claimed in claim 1, further comprising at least one emulsifier.

16. The preparation as claimed in claim 1, wherein said preparation is a medical preparation.

17. The preparation as claimed in claim 1, wherein said preparation is a cosmetic or dermatological preparation.

18. A method for treating or preventing itchiness, hyperreactive skin conditions, sensitive skin, skin subjected to photostress, or impure skin comprising applying to the skin a topical, anti-inflammatory cosmetic or dermatological preparation comprising at least one compound of the formula 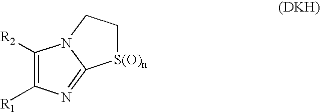
wherein
n is 1 or 2; and
R₁ and R₂, independently of each other, are selected from the group consisting of phenyl, 2-tolyl, 3-tolyl, 4-tolyl, 2-methoxyphenyl, 3-methoxyphenyl, and 4-methoxyphenyl.

19. The method as claimed in claim 18, wherein said applying step comprises applying said preparation to sun-exposed skin.

20. The method as claimed in claim 18, wherein said applying step comprises applying said preparation to the skin to treat or prevent at least one condition selected from the group consisting of psoriasis, atopic eczema, acne, rosacea, allergic dermatitis, irritative contact dermatitis, sunburn, shaving burn, diaper dermatitis, seborrhoeic dermatitis, sun allergy and actinic keratoses.
